(19) United States
(12) Patent Application Publication
(10) Pub. No.: US 2002/0077529 A1
(43) Pub. Date: Jun. 20, 2002

(54) Pneumatic control apparatus for an endoscope

(76) Inventor: Rainer Zotz, Wiesbaden (DE)

Correspondence Address: Jodi-Ann McLane, Salter & Michaelson, 321 South Main Street, Providence, RI, 02903-7128, US

(21) Appl. No.: 09/965,019

(22) Filed: Sep. 27, 2001

(30) Foreign Application Priority Data
Mar. 21, 2000 (DE) DE 100 13 685.0
Mar. 20, 2001 (EP) EP 01 106 906.9

(51) Int. Cl. A61B 1/01

(52) U.S. Cl. 600/152

(57) ABSTRACT

The present invention relates to an apparatus for pneumatic control of an endoscope for ultrasonic examination, the apparatus preferably including a collar, the collar being placed around the endoscope and comprising at least one chamber for receiving a fluid. The apparatus may also include at least one supply for the fluid, and may be provided in combination with an endoscope for ultrasonic examination.

### DESCRIPTION

1. Technical Field

The present invention relates to a pneumatic control apparatus for an endoscope of the type used for ultrasonic examinations.

2. Background of Related Art

As a non-invasive imaging technique, the ultrasonic technique is widely used in medicine for diagnostic purposes because it allows information to be obtained about pathological variations of an organism in a comparatively straightforward manner without surgery.

Since the ultrasonic technique is a sectional view method, which depending on the position of the transducer reproduces only one plane, for a complete picture the transducer has to be moved over the region in question.

An important area of application for ultrasonic diagnostics is transoesophageal echocardiography, for which the transducer is attached to the tip of a flexible endoscope and may be advanced through the oesophagus into the immediate vicinity of the heart.

In order to obtain as complete as possible a mapping of the heart and its surroundings, in particular of the coronary vessels, it is necessary for the transducer to be able to be brought into as many different positions as possible. This is currently achieved by a manual control at the top of the endoscope or through electronic displacement. The drawback of electronic control is that rotation is currently possible only in one plane.

Thus, for the currently used multi-plane transoesophageal echocardiography (multi-plane TEE) the transducer is disposed in a perpendicular manner on the side of the endoscope and rotates in said perpendicular x-axis. Although with the known control mechanisms extensive parts of the heart may be detected, it has been shown that the existing control options are less than satisfactory for specific regions. With said options, for instance, it is not possible to detect the left coronary artery (riva) and the right coronary artery in a reproducible manner.

There is therefore a need for further control options in order to be able to bring the transducer into additional planes and hence also render accessible regions, which up till now have been either not detectable or only inadequately detectable.

### SUMMARY

According to the invention the object is achieved by an apparatus for pneumatic control of an endoscope, in the form of a collar, which is placed around the endoscope, wherein the collar comprises at least one chamber for receiving a fluid as well as at least one supply for the fluid.

According to a further aspect the invention, an apparatus for pneumatic control of an endoscope is provided, wherein the apparatus is comprised of at least one annular collar for embracing the endoscope and which comprises at least one inflatable chamber for receiving a fluid as well at least one supply for the fluid.

According to a further aspect the invention, an apparatus having four chambers forming an annular structure is provided. Two of the four chambers being longer than the two other chambers, the two longer chambers being situated opposite to each other having on both sides one of the two shorter chambers, wherein each of the shorter chambers are situated at an opposite end of the longer chambers, and wherein each of the chambers is provided with a supply for receiving a fluid.

According to yet a further aspect the invention, an endoscope for ultrasonic examinations comprising a shaft and a transducer is provided. The endoscope for ultrasonic examination comprises an apparatus for pneumatic control, the apparatus including at least one collar comprising at least one inflatable chamber for receiving a fluid as well as at least one supply for the fluid, the collar being preferably annular.

According to a preferred embodiment, the invention relates to an endoscope for ultrasonic examinations as set forth above, wherein the apparatus is comprised of four chambers, the chambers being separate from one another, and each chamber being provided with a supply for supplying a fluid.

According to yet a further aspect the invention, the use of an apparatus for pneumatic control of an endoscope for ultrasonic examinations is disclosed, wherein the apparatus is comprised of at least one collar for embracing the endoscope, the collar having an annular and/or cap-like construction, wherein the at least one collar comprises at least one inflatable chamber for receiving a fluid, as well as at least one supply for the fluid.

In use, when the chamber or some of the chambers are charged with the fluid, the chamber or chambers expand and exert pressure on the endoscope and/or the area around the endoscope, with the result being that the position of the endoscope and/or of the transducer changes.

The collar may comprise one or more chambers. The chambers may be separated, or individual chambers may be selectively connected to one another so that a flow of fluid between the connected chambers is possible.

The chamber or chambers may form a ring, which embraces the endoscope body, or have a cap-like construction for receiving the tip of the endoscope. The apparatus may comprise two or more rings, which may be optionally combined with the cap-like form of construction.

The individual chambers can be formed by dividing an annular tube into the desired number of chambers, e.g. by providing a suitable separation device between two adjacent chambers such as a seam or the like. Further, individual ballons can be linked together by sealing, welding, stitching or the like as would be known to one of skill in the art.

According to need, the chambers or balloons can be different in size, for example in volume, length, diameter etc. They can have a ball-like shape or more cylindrical shape. That is, the specific design of the apparatus, in particular the chambers building the apparatus, is not particularly restricted and can be chosen according to need as would be known to one of skill in the art.

The apparatus can be moved along the endoscope to bring it in a position best suited for imaging. For use the apparatus is put on the endoscope in the desired position.

Depending on the position of the apparatus on the endoscope and the number and arrangement of the chambers it is possible, depending on the quantity of fluid supplied to a chamber, to control the pressure and/or direction of pressure exerted by the individual chambers and hence bring the transducer into correspondingly different positions.

In the simplest case, the exertion of pressure is used to increase the distance of the transducer from the heart, thereby also increasing the sound angle and enabling correspondingly extended mapping.

The operation of increasing the distance is usually referred to in the art as “introducing a stand-off-pad”.

The fluid is introduced into the chambers via a line, wherein each or individual chambers can be provided with a separate supply. Chambers which are separate from one another each have a separate supply while chambers which are fluidly connected may share a supply.

The nature of the fluid is not critical for the mode of operation of the apparatus according to the invention. Since in the case of the presently described application the endoscope with the collar is introduced into the oesophagus, the fluid should be medically safe and should moreover not impede the ultrasound. A fluid, which meets the requirements described above, is, for example, water.

The material of the apparatus, when the latter is charged with the fluid, has to be expandable enough to enable the desired pressure to be exerted, i.e. the material has to be sufficiently elastic or compliant, and preferably should be able to contract again during draining of the fluid, and it has to be strong enough to ensure that the apparatus does not tear. Furthermore, the material should be medically safe.

Said requirements regarding elasticity and strength may vary depending on the actual application.

Suitable materials are e.g. plastics materials such as are currently in general use in medical technology and which meet the above criteria as would be known to those of skill in the art.

### BRIEF DESCRIPTION OF THE DRAWINGS

It should be understood that the drawings are provided for the purpose of illustration only and are not intended to define the limits of the invention. The foregoing and other objects and advantages of the embodiments described herein will become apparent with reference to the following detailed description when taken in conjunction with the accompanying drawings in which:

FIG. 1 shows the various axes, about which a TEE probe may be moved;

FIG. 2 shows the sound cone of a TEE probe according to FIG. 1;

FIG. 3 is a preferred construction of the apparatus according to the invention;

FIG. 4 is a further preferred construction of the invention;

FIG. 5A is a front view of a preferable construction of the apparatus according to the invention; and

FIG. 5B is a side view of a preferable construction of the apparatus according to the invention.

### DETAILED DESCRIPTION OF THE ILLUSTRATIVE EMBODIMENTS

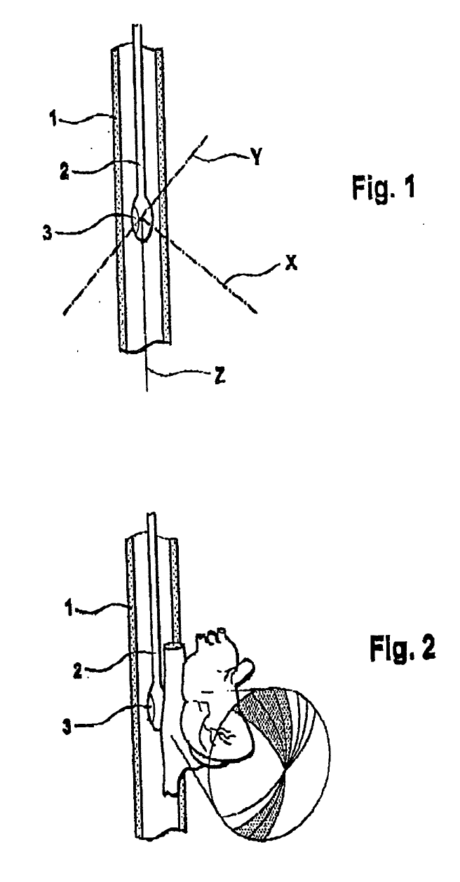

FIG. 1 shows the possible axes, about which a TEE probe 2 may be moved, wherein the z-axis extends parallel to the longitudinal axis of the probe 2 and/or of the oesophagus 1 (gullet), the x-axis is the axis which emanates perpendicular to the z-axis from the transducer 3 and about which the transducer 3 rotates for a 3D image, and the y-axis is at right angles to the other two axes, i.e. in relation to the diagram, perpendicular to the drawing plane.

FIG. 2 shows the sound angle of a currently common transoesophageal representation of the heart as a total organ, wherein the transducer 3, which is disposed at right angles to the side of the endoscope or gastroscope 2, rotates in the perpendicular x-axis.

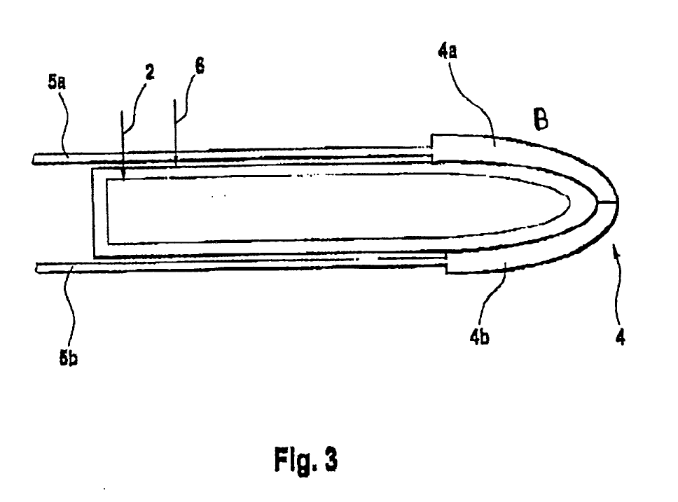

The form of construction B shown in FIG. 3 for an apparatus 4 according to the invention for pneumatic control of an endoscope 2 or gastroscope embraces the tip of the endoscope 2 like a cap.

In FIG. 3 the illustrated cap-like apparatus 4 includes two separate chambers 4a, 4b for receiving a fluid, which is supplied to the individual chambers 4a, 4b via supply 5a, 5b.The construction B may be used to control, in particular, the tip of the instrument.

The endoscope here is moreover provided with a covering 6, such as is generally used for reasons of hygiene. The portion of an endoscope shown in FIG. 3 usually has a length of around 50 to 80 cm and a diameter of 1 cm although the dimensions may be varied as would be known to one of skill in the art.

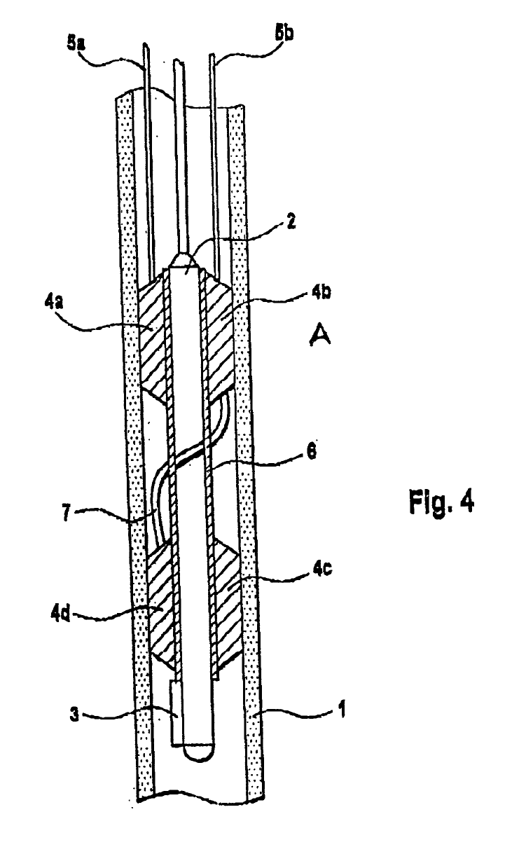

FIG. 4 shows a further construction A of the invention, in which the apparatus 4 does not surround the tip of the endoscope but is applied in an annular manner around the endoscope body. In each case two chambers 4a, 4b and 4c, 4d lying opposite one another embrace the endoscope body so as to form two rings at different levels.

The two upper chambers 4a, 4b in the drawing each have a supply 5a, 5b for supplying the fluid. Charging of the two lower chambers 4c, 4d in the drawing is effected via connecting lines 7. For the embodiment of FIG. 4, the chambers lying opposite and at different levels to one another, namely 4a, 4c and 4b 4d, are connected to one another. In FIG. 4 only the connection between the chambers 4b and 4d is shown the connection of chambers 4a and 4c being the same.

In the construction of the invention shown in FIG. 4, the chambers are disposed on the covering 6 and may be separately formed and connected to the covering 6 or be formed as a unitary component with the covering 6.

In principle, the number of chambers 4a, b etc., their size, shape and their arrangement on the endoscope may be selected in any desired manner as would be known to those of skill in the art. For example, the chambers may be disposed at one level only or alternatively at more than two different levels, at the same time one or more chambers, which embrace the tip e.g. in the manner shown FIG. 3, may be provided.

All or some of the chambers may be disposed on covering 6 and/or be connected to the covering. The covering 6 or an additional covering may be drawn over the apparatus 4 to sheath part or all of the apparatus 4.

The nature of the fluid supply may also be selected in accordance with the requirements and purpose for each case. In principle, each chamber may be provided with a separate supply 5a, b. Individual chambers may communicate with one another e.g. via connecting lines 7, and may have a single common supply.

The supply 5a, 5b may run within the endoscope. In such a case a connection hole in the wall of the endoscope to allow the supply to be connected with the interior of the apparatus 4, or chamber to be provided with fluid, is preferably provided.

According to a further construction, the apparatus 4 in the application situation may, in the filled state, form a balloon which encloses the tip of the endoscope including the transducer 3. This construction is particularly suitable for taking images of the heart from the stomach. To this end, the endoscope 2 plus transducer 3, which is provided with the apparatus 4 is introduced into the stomach and there the apparatus 4 is filled with fluid. The apparatus 4 surrounding the front end of the endoscope 2 and transducer 3 expands like a balloon and in this manner, enables the introduction of a suitable stand-off-pad.

The tip of the probe is in this instance positioned in the stomach in such a way that the heart may be exposed to ultrasonic waves through the diaphragm. The construction as a balloon may also be used in combination with the previously described suitable constructions.

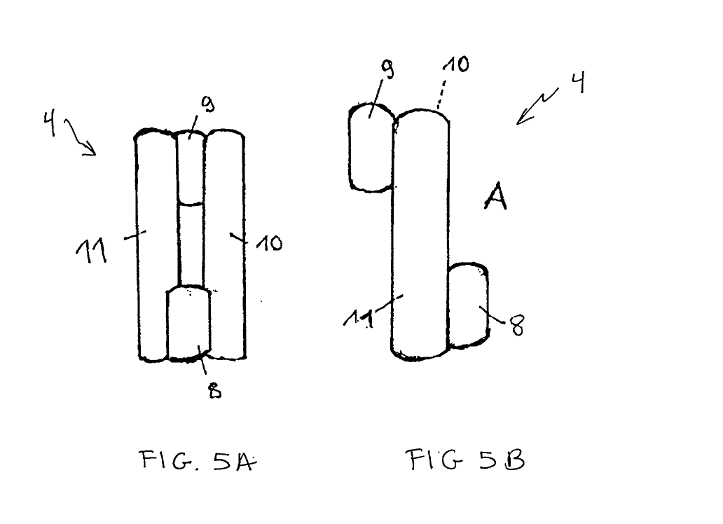

FIGS. 5A and 5B show a front and side view of a particularly preferred embodiment of the apparatus. In this embodiment, the apparatus 4 includes four individual chambers 8, 9, 10, 11 which are linked with each other to form an annular structure. In use, this annular structure is put on an endoscope to embrace the endoscope.

The apparatus of this embodiment preferably comprises two longer chambers 10, 11 and two shorter chambers 8, 9. The two shorter chambers 8, 9 each being preferably positioned between the longer chambers 10, 11 to separate the longer chambers 10, 11 from each other. In this embodiment, each of the shorter chambers 8, 9 is preferably located at an opposite end of the longer chambers 10, 11.

The longer chambers 10, 11 may have a length in the range of from 5 to 10 cm and the shorter chambers 8, 9 of from 1 to 2 cm, the diameter of both may vary in the range of from 1 to 4 cm. However it is to be understood that the length as well as the diameter may vary from said ranges, if desired or needed, as would be known to one of skill in the art.

The embodiment of FIGS. 5A and 5B is particularly suited for obtaining improved ultrasonic images of the heart. For obtaining qualitatively good and diagnostically useful images the transducer 3 has to be pressed against the oesophagus 1 which is done by bending the transducer into the direction of the heart. By this movement the oesophagus 1 is pushed into the direction of the left ventricle of the heart (the ventricle next to the oesophagus), and the mediastinal connective tissue is compressed resulting in good conditions for a homogenous spreading of the ultrasonic sound.

In order to provide the above advantageous conditions, the embodiment shown in FIG. 5 is applied to the endoscope in such a manner that the short chamber 8 (front chamber) is in a position in front of the transducer (Ultrasonicflash). In this case, the second short chamber 9 (rear chamber) is directed towards the mouth of the patient. By filling completely and/or partly the chambers, or individual chambers, the desired motion of the transducer can be obtained.

By filling the front chamber 8 it is possible to elongate the stand-off-pad of the ultrasonic waves. Filling the rear chamber 9 enhances the retroflexion of the transducer 3 and allows a sound angle sloping into the direction of spreading of the ultrasonic sound.

The long chambers 10, 11 having the form of a cylinder are positioned on the left and right side when applied to the endoscope and serve to stabilize the apparatus and to prevent lateral bending of the flexible endoscope.

For individually filling the chambers 8, 9, 10, 11 with a suited fluid, e.g. water, each chamber is provided with a supply (not shown). Preferably, the supplies may run along the outside the tube of the endoscope and leave the oesophagus through the mouth of the patient and may be connected to a manometer. They can be fixed to the tube by any suitable device such as rubber rings. With respect to further arrangement and design, in principle the same is valid as set out above with respect to the other embodiments of the apparatus.

According to a further embodiment, the invention encompasses an endoscope for ultrasonic examination comprising an apparatus as disclosed above as well as the use of such an apparatus for pneumatic control of an endoscope suited for ultrasonic examination.

Because of the many different ways of constructing and arranging the apparatus according to the invention for pneumatic control of an endoscope, the range of possible movements of such probes may be markedly widened.

It will be understood that various modifications may be made to the embodiment disclosed herein. Therefore, the above description should not be construed as limiting, but merely as exemplifications of a preferred embodiment. Those skilled in the art will envision other modifications within the scope spirit of the invention.

What is claimed is:

1. An apparatus for pneumatic control of an endoscope used in ultrasonic examinations, comprising:
at least one annular collar constructed and arranged to embrace the endoscope during use, the annular collar having at least one inflatable chamber constructed and arranged to receive a fluid; and
at least one supply constructed and arranged to supply the fluid to the inflatable chamber.

2. The apparatus according to claim 1, wherein the at least one annular collar is moveable along a shaft of the endoscope during use.

3. The apparatus according to claim 1, wherein the at least one inflatable chamber comprises at least two chambers which are separated from one another such that they are fluidly independent of each other.

4. The apparatus according to claim l,wherein the annular collar is a combination selected from the group consisting of at least two annular collars and at least one annular collar and one collar of cap-like construction.

5. The apparatus according to claim 1, wherein the annular collar is comprised of four chambers, two of the four chambers having a length greater than the two other chambers, the two longer chambers being situated opposite to each other having on both sides one of the two shorter chambers, wherein each of the shorter chambers is situated at an opposite end of the longer chambers, and wherein each of the chambers is provided with a supply constructed and arranged to supply a fluid to the chambers.

6. An apparatus for pneumatic control of an endoscope used in ultrasonic examinations, comprising:
a cap-like member constructed and arranged to surround a tip of the endoscope, the cap-like member including at least one inflatable chamber.

7. The apparatus according to claim 6, wherein the at least one inflatable chamber comprises at least two chambers which are separated from one another such that they are fluidly independent of each other.

8. The apparatus according to claim 6, wherein the cap-like member comprises at least one annular collar and one collar of cap-like construction.

9. The apparatus according to claim 8, wherein the at least one annular collar and the one collar of cap-like construction are fluidly connected to each other by a connecting line.

10. An endoscope for use in ultrasonic examinations comprising a shaft and a transducer, the endoscope comprising:
an apparatus constructed and arranged with pneumatic control, the apparatus having at least one collar including at least one inflatable chamber constructed and arranged to receive a fluid; and
at least one supply constructed and arranged to deliver the fluid to the at least one inflatable chamber.

11. The endoscope for ultrasonic examinations according to claim 10, wherein the at least one inflatable chamber comprises four chambers divided from one another such that they are fluidly independent of each other, each of the four chambers being provided with a supply constructed and arranged to supply a fluid to each of the chambers.

12. The endoscope for ultrasonic examinations according to claim 10, wherein the apparatus is movable along the endoscope.

13. A method of using an apparatus for pneumatic control of an endoscope for ultrasonic examinations, comprising the steps of:
providing at least one collar having at least one inflatable chamber constructed and arranged to receive a fluid;
supplying the fluid to the at least one inflatable chamber so as to at least partially inflate the chamber;
embracing the endoscope with the collar having the at least partially inflated chamber.

14. The method according to claim 13, wherein the at least one collar has an annular structure.
